(19) United States
(12) Patent Application Publication
(10) Pub. No.: US 2015/0147410 A1
(43) Pub. Date: May 28, 2015

(54) Wound Dressing Comprising Bio-Cellulose and Silver Nanoparticles

(71) Applicant: Novatec Healthcare Company Limited, Samutprakan (TH)

(72) Inventors: Adisorn Apasuthirat, Samutprakarn (TH); Sombat Rungsilp, Phatthalung (TH)

(21) Appl. No.: 14/402,503

(22) PCT Filed: Jan. 29, 2013
(86) PCT No.: PCT/TH2013/000004

§ 371 Date: Nov. 20, 2014

(30) Foreign Application Priority Data
May 21, 2012 (TH) 1201002468

(51) Int. Cl. A61L 15/46; B22F 1/00; C22C 5/06; A61L 15/28

(52) U.S. Cl. CPC A61L 15/46; A61L 15/28; B22F 1/0007; C22C 5/06; A61L 2400/04; A61L 2300/404; A61L 2300/104
USPC 424/618; 420/501

(57) ABSTRACT

The present disclosure relates to dressings for wounds, methods of preparing thereof and methods of using thereof. Also, the present disclosure relates to a wound dressing comprising: bio-cellulose; and silver nanoparticles, wherein the concentration of silver nanoparticles is about 1000 μg/100 cm2 or less or about 1000 μg/cm3 or less, and wherein the silver nanoparticles have a localized surface Plasmon resonance maxima of about 600 nm to about 800 nm.

### TECHNICAL FIELD

The present disclosure relates to dressings for wounds, methods of preparing thereof and methods of using thereof. Specifically, the disclosure provides a cellulose wound dressing with antimicrobial properties.

### BACKGROUND

Various techniques and dressings are used in the treatment of wounds. The type of technique and dressing is dependent on the goal of the treatment and the type of wound being treated. Goals of treatment may include pain reduction, compression, immobilization, protection from infection or further injury, promotion of healing, and scar minimization. Wounds include, for example lacerations, burns, atraumatic wounds and traumatic wounds.

Treatment of wounds involves evaluation, investigation, closure (if necessary and possible), and management. Management often involves cleansing and dressing of the wound in a manner that promotes expedient healing, preferably with minimal scarring. Dressing selection for management of a wound is dependent upon the type of wound, the amount of wound exudates, the location and size of the wound, and the level of support needed (e.g., how adhesive to the wound must the dressing be).

Management may also involve the application of antimicrobial agents for the prevention of infection during wound healing. These can include, for example, alcohol, peroxides, silver, iodine, antibiotics, and the like. The moist environment formed by wound exudates and moist dressings used to prevent scaring and wound exacerbation typically promotes microbial growth. Microbial growth at a wound site can lead to an infection of the wound. Thus, the use of an antimicrobial agent is often beneficial for preventing infections during wound healing.

Wounds can include, for example, acute and/or chronic wounds, wounds in individuals with co-morbidities, burns, recurrent wounds, tunneling wounds, complicated wounds and the like, and wounds that require additional attention. These types of wounds can require upkeep to prevent infection and insure proper healing. Dressings must be changed frequently for debridement of the wound, to provide a clean surface and to maintain antimicrobial status of the wound. However, changing of dressings can also cause pain, induce inflammation at the wound site, and expose the wound to the surrounding environment, which may contain microbes. Thus, the frequency of dressing changes must be balanced against pain and inflammation caused by dressing changes and also against exposure of the wound to environments that cause infection.

Accordingly, there is a need for a wound dressing that affords a moist environment, absorbs exudates, and provides antimicrobial treatment to the wound. Furthermore, there is a need for a wound dressing capable of providing an indication of when the wound dressing should be changed or is no longer providing antimicrobial benefits to the wound.

### SUMMARY

The present disclosure provides a wound dressing including bio-cellulose; and silver nanoparticles.

In a first aspect, there is provided a wound dressing comprising: bio-cellulose; and silver nanoparticles, wherein the concentration of silver nanoparticles is about 1000 μg/100 cm² or less, about 1000 μg/cm³ or less or a combination thereof, and wherein the silver nanoparticles have a localized surface Plasmon resonance maxima of about 600 nm to about 800 nm.

In a second aspect, there is provided a method of treating a wound comprising: providing a blue color wound dressing comprising bio-cellulose and silver nanoparticles, wherein the blue color of the blue wound dressing is imparted by the silver nanoparticles; applying the blue wound dressing to a wound; and removing the blue wound dressing from the wound when the blue wound dressing is no longer blue but instead a color selected from the group consisting of a yellowish color, off-white color, the white color of the bio-cellulose itself and a combination of one or more thereof.

In a third aspect, there is provided a method of preparing a blue wound dressing comprising: preparing bio-cellulose; and, adding a dilute solution of silver nanoparticles to the bio-cellulose to form a blue wound dressing, wherein the concentration of silver nanoparticles in the blue wound dressing is about 1000 μg/100 cm² or less, about 1000 μg/cm³ or less or a combination thereof, and wherein the silver nanoparticles impart a blue color to the wound dressing.

In a fourth aspect, there is provided a wound dressing comprising a hydrogel comprising: carboxymethyl cellulose; and silver nanoparticles, wherein the concentration of silver nanoparticles is about 1000 μg/cm³ or less, and wherein the silver nanoparticles have a localized surface Plasmon resonance maxima of about 600 nm to about 800 nm.

In another aspect, the concentration of silver nanoparticles in the wound dressings of the present disclosure is from about 50 μg/100 cm² to about 1000 μg/100 cm² or from about 50 μg/cm³ to about 1000 μg/cm³.

The foregoing summary is illustrative only and is not intended to be in any way limiting. In addition to the illustrative aspects, embodiments, and features will become apparent by reference to the drawings and the following detailed description.

### BRIEF DESCRIPTION OF THE DRAWINGS

Embodiments of the disclosure are described herein with reference to the drawings in which:

FIG. 1 depicts cellulose fibers produced by plant matter.

FIG. 2 depicts a top down view of a bio-cellulose sheet in accordance with the present disclosure.

FIG. 3 depicts a horizontal view of a bio-cellulose sheet in accordance with the present disclosure.

FIG. 4A depicts silver nanospheres at a 100 nm scale that have a localized surface Plasmon resonance (LSPR) in the pale yellow region (at a wavelength of about 400 nm in the visible region, the silver nanospheres absorb violet light and subsequently scatter pale yellow light).

FIG. 4B depicts silver nanodisks at a 100 nm scale that have a LSPR in the darker yellow region (at a wavelength of about 475 nm in the visible region, the silver nanodisks absorb dark blue light and subsequently scatter darker yellow light).

FIG. 4C depicts silver nanodisks at a 100 nm scale that have a LSPR in the red region (at a wavelength of about 550 nm in the visible region, the silver nanodisks absorb green light and subsequently scatter red light).

FIG. 4D depicts silver nanoplates at a 100 nm scale that have a LSPR in the violet region (at a wavelength of about 570 nm in the visible region, the silver nanoplates absorb green-yellow light and subsequently scatter violet light.

FIG. 4E depicts silver nanoplates at a 200 nm scale that have a LSPR in the dark blue region (at a wavelength of about 600 nm in the visible region, the silver nanoplates absorb orange light and subsequently scatter dark blue light).

FIG. 4F depicts silver nanoplates at a 200 nm scale that have a LSPR in the mid blue region (at a wavelength of about 650 nm in the visible region, the silver nanoplates absorb red-orange light and subsequently scatter mid blue light).

FIG. 4G depicts silver nanoplates at a 200 nm scale that have a LSPR in the light blue region (at a wavelength of about 750 nm, the silver nanoplates absorb red light and subsequently scatter light blue light).

FIG. 4H depicts silver nanoprisms at a 200 nm scale that have a LSPR in the pale blue region (at a wavelength of about 800 nm, the silver nanoprisms absorb infrared light and subsequently scatter pale blue light (e.g., strongly scatter pale blue light)).

FIG. 5 depicts three different angles on triangular silver nanoparticles of the present disclosure.

FIG. 6A depicts how a wound dressing of the present disclosure appears when loosely packed into a wound.

FIG. 6B depicts the wound dressing of FIG. 6A after the silver nanoparticles having an LSPR in the blue range are released from the wound dressing.

FIG. 6C depicts the removal of the wound dressing of FIG. 6B;

FIG. 6D depicts the wound of FIG. 6A after removal of the dressing of 6B;

FIG. 7 depicts the antimicrobial activity of the blue silver nanoparticles of the present disclosure against several examples of wound pathogens.

FIG. 8 depicts a graph of the level of patient reported pain using a calcium alginate wound dressing or a wound dressing of an embodiment of the present disclosure;

FIG. 9A depicts a cavity wound prior to treatment with the wound dressing of the present disclosure;

FIG. 9B depicts the cavity wound of FIG. 9A following 14 days of treatment with the wound dressing of the present disclosure;

FIG. 9C depicts the cavity wound of FIG. 9A following 28 days of treatment with the wound dressing of the present disclosure;

FIG. 9D depicts the cavity wound of FIG. 9A following 39 days of treatment with the wound dressing of the present disclosure;

FIG. 10A depicts a dog bite wound prior to treatment with a wound dressing in accordance with the present disclosure;

FIG. 10B depicts the dog bite wound of FIG. 10A following 5 days of treatment with a wound dressing in accordance with the present disclosure;

FIG. 10C depicts the dog bite wound of FIG. 10A following 12 days of treatment with a wound dressing in accordance with the present disclosure;

FIG. 10D depicts the dog bite wound of FIG. 10A following 20 days of treatment with a wound dressing in accordance with the present disclosure;

FIG. 11A depicts a diabetic foot ulcer covered by a callous prior to treatment with a wound dressing in accordance with the present disclosure;

FIG. 11B depicts the diabetic foot ulcer of FIG. 11A following 14 days of treatment with a wound dressing in accordance with the present disclosure;

FIG. 11C depicts the diabetic foot ulcer of FIG. 11A following 21 days of treatment with a wound dressing in accordance with the present disclosure; and,

FIG. 11D depicts the diabetic foot ulcer of FIG. 11A following 42 days of treatment with a wound dressing in accordance with the present disclosure.

### DETAILED DESCRIPTION

The illustrative embodiments described in the following detailed description, drawings, and claims are not meant to be limiting. Other embodiments can be utilized, and other changes can be made, without departing from the spirit or scope of the subject matter presented herein.

Unless specified otherwise, the terms “comprising” and “comprise” as used herein, and grammatical variants thereof, are intended to represent “open” or “inclusive” language such that they include recited elements but also permit inclusion of additional, un-recited elements.

As used herein, the term “about”, in the context of concentrations of components of the formulations, conditions, other measurement values, etc., means+/−5% of the stated value, or +/−4% of the stated value, or +/−3% of the stated value, or +/−2% of the stated value, or +/−1% of the stated value, or +/−0.5% of the stated value, or +/−0% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present disclosure provides wound dressings that can afford a moist environment, absorb exudates, inhibit scarring, facilitate debridement of wounds, and provide antimicrobial treatment to a wound. Further, the present disclosure provides a self-indicating wound dressing that can provide an visual indication of when the wound dressing should be changed and/or is no longer providing antimicrobial benefits to the wound. The visual indication of when the wound dressing should be changed and/or is no longer providing antimicrobial benefits includes a change in the color of the self-indicating wound dressing. In some embodiments, the wound dressings can include a bio-cellulose sheet in combination with silver nanoparticles. In other embodiments, the wound dressing can include a gel (e.g., bio-cellulose colloid gel, bio-cellulose gel, and/or hydrogel) in combination with silver nanoparticles. The present disclosure further provides methods of preparing and using the wound dressings of the disclosure.

The wound dressings of the present disclosure that include the combination of the bio-cellulose (e.g., bio-cellulose sheet, bio-cellulose colloid gel, or bio-cellulose gel) with the silver nanoparticles (in a concentration of 1000 μg/100 cm² or less in the case of the bio-cellulose sheet and in a concentration of 1000 μg/cm³ or less in the case of the bio-cellulose colloid gel or bio-cellulose gel) provide a surprising synergistic effect with respect to healing wounds more effectively and faster than known wound dressings.

Bio-Cellulose

In accordance with the present disclosure, bio-cellulose is cellulose produced by bacteria. Intracellular biological synthesis of cellulose occurs via many organisms, such as, for example, Vallonia (algae), Saprolegnia, Dictystelium, Discodium (fungi), Aerobacter, Agrobacterium, Pseudomonas, Rhizobium, Alcaligenes, Saecina, and Zoogloea (bacteria). However, Acetobacterxylinum (A. xylinum), is capable of synthesizing fibrous cellulose extracellularly and, therefore, is able to produce a bio-cellulose that is more readily usable.

The morphological characteristics of cellulose produced by A. xylinum (also known as Gluconoacetobacter xylinus) depends in large part on the conditions (static, agitated, etc.) and media in which the A. xylinumis grown. The specific medium on which the A. xylinumis grown also has a great impact on, among other things, the crystallinity of the cellulose produced. While A. xylinum is unable to metabolize some sugars, such as, for example, xylose, it is capable of metabolizing other sugar alcohols, such as, for example, arabitol, with a productivity level much higher than metabolism of glucose. The pH of the media, the carbon source for the A. xylinum, and acidity of the byproducts of metabolism of the carbon source by the A. xylinum all impact the resulting cellulose.

In accordance with the present disclosure, bio-cellulose can be produced or biosynthesized by A. xylinum. Generally, bio-cellulose can be produced or biosynthesized by A. xylinum in any media. In embodiments, the medium can contain a source of sugar or alcohol. In embodiments, the medium can contain can contain one or more of coconut juice, pineapple juice, broken-milled rice and yeast extract. The bio-cellulose can be in the form of fibrils or fibers. The fibrils or fibers can have a diameter of from about 3 nm to about 8 nm. The fibrils or fibers can be bundled into fiber bundles. The fiber bundles can have a diameter of from about 100 nm to about 150 nm. In embodiments, the bio-cellulose can have a white color.

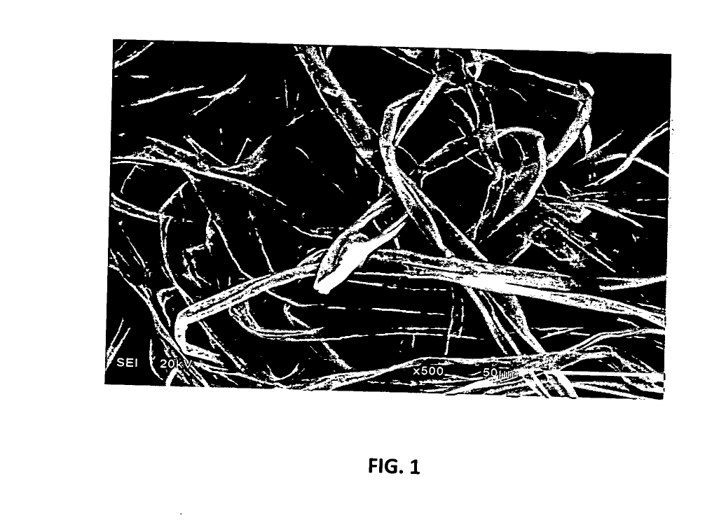

A. xylinum produces a very fine cellulose fiber network, much finer than that of plant cellulose depicted in FIG. 1. Bacterial cellulose from A. xylinum exhibits a highly crystalline structure affording it a high tensile strength, elasticity and modulus. It also has a high level of mechanical stability. A. xylinum cellulose can retain up to 200 times its dry weight in water. It exhibits pseudoplastic viscosity similar to xanthan gels, which is not diminished at high temperatures or low shear rates.

In embodiments, the bio-cellulose can be grown in a sanitized covered tray using A. xylinum present at a concentration of about 10⁷ to about 10⁸ cfu/ml. In embodiments, the dimensions of the sanitized covered tray can be dependent on the dimensions of the bio-cellulose required. In embodiments, the dimensions of the sanitized covered tray can be modified based on the desired or required dimensions of the bio-cellulose. In embodiments, the bio-cellulose can be cultured using A. xylinum over a period of about 7 to about 12 days. In embodiments, the bio-cellulose can be cultured using a pH condition of about 4.5 to about 5 and a temperature condition of about room temperature. In embodiments, the bio-cellulose can be cultured using a temperature condition of about 28° C. to about 34° C. The cover of the sanitized covered tray can protect the bio-cellulose from contaminants in the surrounding outside environment.

In embodiments, under the foregoing conditions, after the bio-cellulose has grown to a thickness of about 0.1 to about 1 cm, the bio-cellulose can be harvested from the sanitized covered tray. The harvested bio-cellulose can then be chemically and physically processed to remove any media, microbial contaminants, and other contaminants. In embodiments, the harvested bio-cellulose can be irradiated to sterilize the bio-cellulose prior to use.

In embodiments, the bio-cellulose is produced or biosynthesized as bio-cellulose fibers having a diameter of about 3 to about 8 nanometers. In embodiments, the bio-cellulose can be formed into fiber bundles having a diameter of about 100 to about 150 nanometers. In embodiments, during synthesis or biosynthesis, the fiber bundles can be formed into a non-woven, multi-layered, three-dimensional sheet structure.

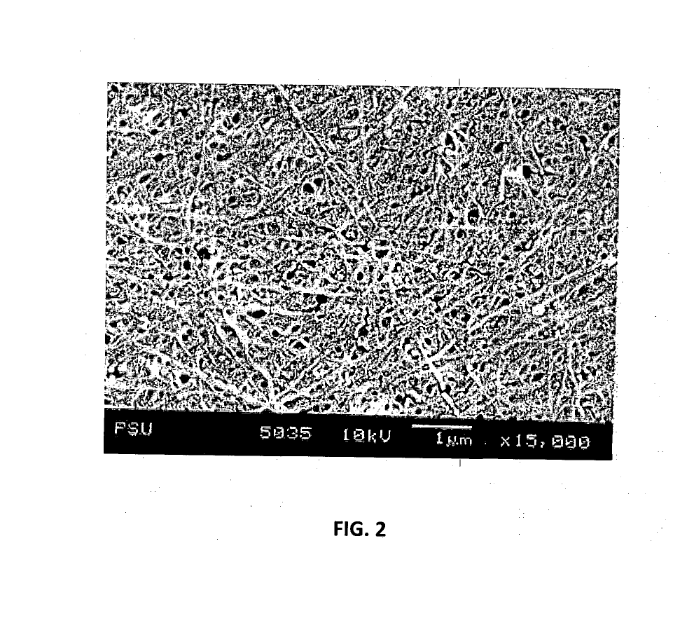

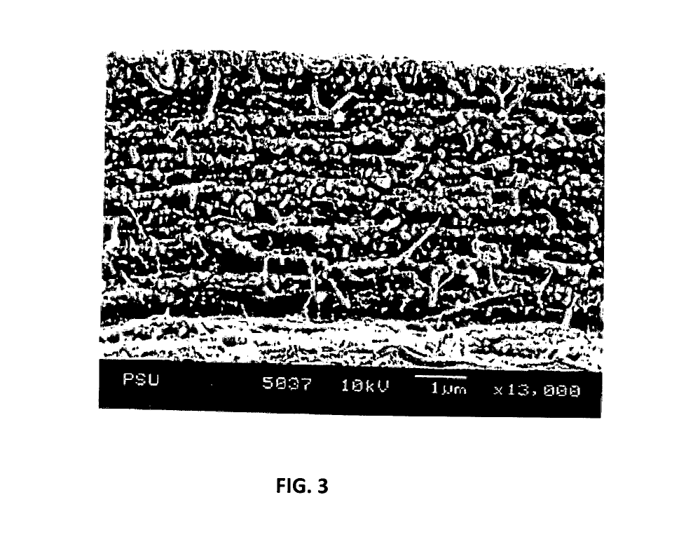

In some embodiments, the bio-cellulose fiber bundles can be formed into a non-woven sheet as depicted in FIG. 2 and FIG. 3. Any number of sheets of bio-cellulose can be layered to provide a bio-cellulose sheet with a desired thickness. In embodiments, the bio-cellulose sheet can have 1 layer or more. In some embodiments, the bio-cellulose sheet can have 3 layers or more. In some embodiments, the bio-cellulose sheet can have 100 layers or more.

In embodiments, the bio-cellulose non-woven, multi-layered, three-dimensional sheet structure provides numerous inter-fiber spaces that allow the bio-cellulose sheet to hold water of more than 200 times the dry weight of the bio-cellulose sheet. These inter-fiber spaces also make the bio-cellulose sheet breathable and provide nanocapillary forces on the bio-cellulose sheet's contacting surface. These nanocapillary forces provide an auto-debridement effect on the bio-cellulose sheet's contacting surface that results in the removal of dead tissue and other foreign matter from the wound site.

In embodiments, after the harvested bio-cellulose is chemically and physically processed, sanitized water can be used to fill inter-fiber spaces of the harvested bio-cellulose. The bio-cellulose can provide moisture to the wound site, allow for the growth of new cells, and consequently allow for wounds to heal faster.

In embodiments, the bio-cellulose sheet dimensions (e.g., thickness, length, and width) can be tailored to the type of wound, size of wound, and/or commercial requirements. In embodiments, the bio-cellulose sheet can have a 3-dimensional shape with about a 0.1 cm to about 1 cm thickness, about a 3 cm to about 100 cm length, and about a 1 cm to about 60 cm width. Other dimensions for the bio-cellulose sheet are also contemplated.

In some embodiments, the wound dressing can include a gel (e.g., bio-cellulose colloid gel, bio-cellulose gel, and/or hydrogel) in combination with silver nanoparticles. In some embodiments, the bio-cellulose fiber bundles can be blended with a gelling agent to form a gel. Gelling agents can include, for example, carboxymethyl cellulose (CMC), xanthan gum and other natural gums, starch, pectin, agar agar, gelatin, alginates, and the like and combinations of these.

In embodiments, a gel can include about 18-25 weight percent (wt %) of bio-cellulose fiber bundles. In embodiments, a gel can include about 0.5-2 wt % of carboxymethyl cellulose, about 1-2 wt % of xanthan gum and/or other natural gums, about 2-20 wt % of starch, about 0.5-1 wt % of pectin, about 0.5-1 wt % of agar agar, about 0.5-7 wt % of gelatin, about 1-5 wt % of alginates, and/or combinations thereof. The remaining components of the gel can include water and silver nanoparticles.

In embodiments, a hydrophilic bio-cellulose colloid gel can be prepared by combining bio-cellulose fiber bundles and carboxymethyl cellulose. In embodiments, a hydrophilic bio-cellulose colloid gel can be prepared by combining 12-20 weight percent (wt %) of bio-cellulose fiber bundles and 0.1-2 wt % of carboxymethyl cellulose. The remaining components of the lipophilic bio-cellulose colloid gel can include water and silver nanoparticles.

In embodiments, a lipophilic bio-cellulose colloid gel can be prepared by combining bio-cellulose fiber bundles, hard paraffin, petroleum jelly, and glycerin. In embodiments, a lipophilic bio-cellulose colloid gel can be prepared by combining 12-20 weight percent (wt %) of bio-cellulose fiber bundles, 2-5 wt % of hard paraffin, 30-45 wt % of petroleum jelly, and 15-25 wt % of glycerin.

In embodiments, a hydrogel can be prepared by combining carboxymethyl cellulose with silver nanoparticles. In embodiments, a hydrogel can be prepared by combining 0.5-5% carboxymethyl cellulose with water and silver nanoparticles.

In embodiments, the gel (e.g., bio-cellulose colloid gel, bio-cellulose gel, or hydrogel) with the silver nanoparticles can be applied directly to a wound, coated onto a sheet to be applied to the wound, coated onto a gauze to be applied to the wound, or a combination of one or more thereof. In embodiments, the sheet can be non-woven or woven. In embodiments, the gauze can be non-woven or woven.

In accordance with some embodiments, a gel (e.g., bio-cellulose colloid gel, bio-cellulose gel or hydrogel) with silver nanoparticles of the present disclosure and a bio-cellulose sheet with silver nanoparticles of the present disclosure may both be applied to the same wound for treatment of the wound. In accordance with some embodiments, a gel (e.g., bio-cellulose colloid gel, bio-cellulose gel or hydrogel) with silver nanoparticles of the present disclosure and a bio-cellulose sheet with silver nanoparticles of the present disclosure may both be applied concurrently to the same wound for treatment of the wound.

Antimicrobial

The wound dressings of the present disclosure further include an antimicrobial agent in the form of silver nanoparticles. While ionized silver in many forms is antimicrobial, certain structural features have been identified that can greatly enhance the antimicrobial properties of the silver. The size, shape, and dielectric properties of the silver nanoparticles alter its localized surface Plasmon resonance (LSPR) and impact its ionization rate and antimicrobial properties.

In embodiments, the silver nanoparticles of the present disclosure can have a particle size of about 200 nm or less. In embodiments, the silver nanoparticles of the present disclosure can have a size of about 80 nm to about 120 nm. In embodiments, the silver nanoparticles can have a circular disk shape, a hexagonal shape, and/or a truncated triangular shape. In embodiments, the silver nanoparticles of the wound dressing can be truncated triangular nanoplates or nanoparticles.

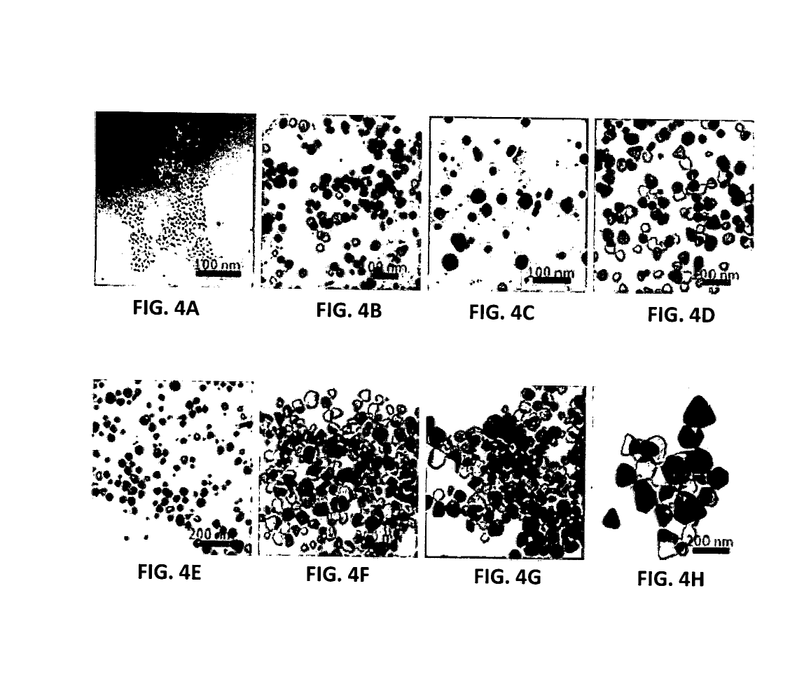

Silver nanoparticles of different sizes are depicted in FIGS. 4A to 4H. The LSPR reflects a different color depending on the particle size and shape of the silver nanoparticle. The silver nanospheres at a 100 nm scale depicted in FIG. 4A have an LSPR of about 400 nm. At a wavelength of about 400 nm in the visible region, the silver nanospheres absorb violet light and subsequently scatter pale yellow light.

The silver nanodisks at a 100 nm scale depicted in FIG. 4B have an LSPR of about 475 nm. At a wavelength of about 475 nm in the visible region, the silver nanodisks absorb dark blue light and subsequently scatter darker yellow light.

The silver nanodisks at a 100 nm scale depicted in FIG. 4C have an LSPR of about 550 nm. At a wavelength of about 550 nm in the visible region, the silver nanodisks absorb green light and subsequently scatter deep red light.

The silver nanoplates at a 100 nm scale depicted in FIG. 4D have an LSPR of about 570 nm. At a wavelength of about 570 nm in the visible region, the silver nanoplates absorb green-yellow light and subsequently scatter violet light.

The silver nanoplates at a 200 nm scale depicted in FIG. 4E have an LSPR of about 600 nm. At a wavelength of about 600 nm in the visible region, the silver nanoplates absorb orange light and subsequently scatter dark blue light.

The silver nanoplates at a 200 nm scale depicted in FIG. 4F have an LSPR of about 650 nm. At a wavelength of about 650 nm in the visible region, the silver nanoplates absorb red-orange light and subsequently scatter mid blue light.

The silver nanoplates at a 200 nm scale depicted in FIG. 4G have an LSPR of about 750 nm. At a wavelength of about 750 nm, the silver nanoplates absorb red light and subsequently scatter light blue light.

The silver nanoprisms at a 200 nm scale depicted in FIG. 4H have an LSPR of about 800 nm. At a wavelength of about 800 nm, the silver nanoprisms absorb infrared light and subsequently scatter pale blue light (e.g., strongly scatter pale blue light).

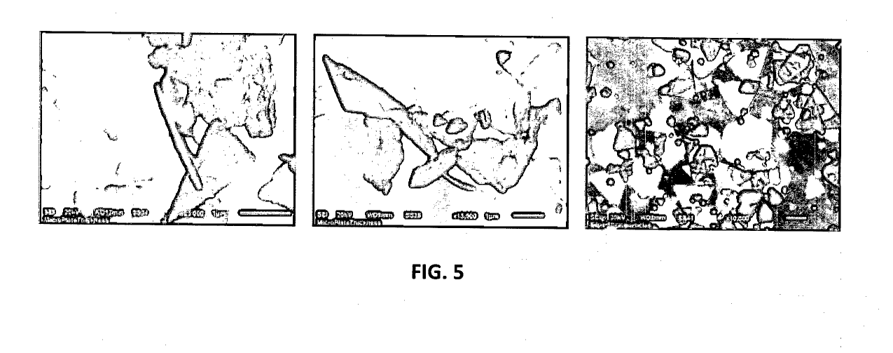

In embodiments, the silver nanoparticles of the present disclosure have an LSPR of about 600 nm to about 800 nm. At a wavelength of about 600 nm to 800 nm, the silver nanoparticles of the present disclosure absorb orange to infrared light and produce a blue color. The silver nanoparticles of the present disclosure impart a blue color to the wound dressing. The blue color produced by the silver nanoparticles can be observed visually by the naked eye, under ambient light illumination or a combination thereof. FIG. 5 depicts silver nanoparticles having a truncated triangular nanoplate shape and/or nanoprism shape and having an LSPR reflecting blue light.

As the silver nanoparticles are ionized and released from the wound dressing into the wound, the visual appearance and/or color of the wound dressing can change. As the silver nanoparticles are ionized and released from the wound dressing into the wound, the nanoparticles can undergo a change in size and/or shape, and can also exhibit other wavelengths including wavelengths outside the visible spectrum.

In embodiments, the wound dressings of the present disclosure can include silver nanoparticles having a broad or somewhat broad particle size distribution that produces or predominantly produces a blue color. In embodiments, the wound dressings of the present disclosure can include a plurality of silver nanoparticles, wherein each silver nanoparticle has a particle size of 200 nm or less, and wherein the plurality of silver nanoparticles produces a blue color. In embodiments, the wound dressings of the present disclosure can include one or more particle sizes of silver nanoparticles that together give rise to a blue color.

As mentioned above, as the silver nanoparticles are ionized and released (e.g., diffused out) from the wound dressing into the wound, the nanoparticles can undergo a change in size and/or shape, and can also exhibit other wavelengths including wavelengths outside the visible spectrum. As such, as the silver nanoparticles are ionized and released from the wound dressing into the wound, the color of the wound dressing changes from the blue color imparted by the silver nanoparticles to a yellowish color, off-white color, the white color of the bio-cellulose itself or a combination of one or more thereof. Thus, after the silver nanoparticles are ionized and diffused out from the wound dressing, the wound dressing is no longer blue thereby providing a visual indication that the wound dressing should be changed and/or is no longer providing antimicrobial benefits.

The silver nanoparticles of the present disclosure display a minimum inhibition concentration (MIC) against wound pathogens of about 1 ppm to about 5 ppm. Wound pathogens include, for example Escherichia coli, Staphylococcus aureus, Acintobacter, Pseudomonas, Streptococcus, Proteus, Klebsiella, Xanthomonas, and the like, and combinations of these. In embodiments, the silver nanoparticles can display a MIC for primary pathogens that infect wounds as shown below:

| | MIC of silver nanoparticles |
|---|---|
| | having a LSPR |
| Microbe | of 600 nm to 800 nm |
| Escherichia coli | 1 ppm |
| Staphylococcus aureus | 2.5 ppm |
| Methicillin-resistant Staphlococcus aureus | 2.5 ppm |
| Acinetobacter baumannii | 1 ppm |
| Pseudomonas aeruginosa | 5 ppm |

Sheet/Gel

In accordance with the present disclosure, a wound dressing of the present disclosure can be in the form of a sheet or a gel. In embodiments, the gel can be a bio-cellulose colloid gel, bio-cellulose gel and/or hydrogel. In embodiments, the gel can be applied directly to a wound. In embodiments, the gel can be applied to a woven sheet, woven gauze, non-woven sheet, and/or non-woven gauze and subsequently applied to a wound. In some embodiments, the gel can be applied to a bio-cellulose sheet that does not contain silver nanoparticles and subsequently applied to a wound.

In embodiments, an antimicrobial bio-cellulose sheet is prepared by: providing a non-diluted solution of silver nanoparticles; diluting the non-diluted solution of silver nanoparticles; and applying the dilute solution of silver nanoparticles to a bio-cellulose sheet. In embodiments, the silver nanoparticles are provided in the non-diluted solution by adding a silver salt, such as, silver nitrate. The use of other silver salts is also contemplated. In embodiments, prior to dilution of the non-diluted solution of silver nanoparticles, the silver nanoparticles can be in a solution of water and a starch or other thickener or stabilizer. The starch can be, for example, modified starch, refined starch, pre-gelatinized starch, and the like, and combinations of these. In embodiments the non-diluted solution of silver nanoparticles can contain about 1000 mg/L of silver nanoparticles.

In embodiments, the non-diluted solution of silver nanoparticles can be diluted prior to use in the wound dressing of the present disclosure. In embodiments, a non-diluted solution containing silver nanoparticles can be diluted to a concentration of about 100 mg/L of silver nanoparticles. The diluting solution can include, for example, water and a humectant. The humectant can be, for example, glycerin and the like. The use of other humectants is also contemplated. In embodiments, the diluting solution can include about 20% to about 40% humectant. In embodiments, the diluting solution can include about 60% to about 80% water. In embodiments, 10 ml or less of the diluted solution or dilute solution of silver nanoparticles (i.e., having a concentration of silver nanoparticles of about 100 mg/L) can be applied to a 100 cm² bio-cellulose wound dressing thereby resulting in a bio-cellulose wound dressing having a concentration of silver nanoparticles of about 1000 μg/100 cm² or less.

In embodiments, a dilute solution of silver nanoparticles is applied to a bio-cellulose sheet and imparts a blue color to the bio-cellulose sheet thereby forming a blue wound dressing. In embodiments, the dilute solution of silver nanoparticles can be applied to coat one or more sides of the bio-cellulose sheet. In embodiments, all sides of the bio-cellulose sheet can be covered with the dilute solution of silver nanoparticles. In some embodiments, the bio-cellulose sheet is saturated with the dilute solution of silver nanoparticles. In embodiments, 10 ml or less of the dilute solution of silver nanoparticles (i.e., having a concentration of silver nanoparticles of about 100 mg/L) can be applied to a 100 cm² bio-cellulose sheet thereby resulting in a bio-cellulose sheet having a concentration of silver nanoparticles of about 1000 μg/100 cm² or less.

In embodiments, the silver nanoparticles are not chemically bound to the bio-cellulose. In some embodiments, the silver nanoparticles in solution are physically retained by the absorptive properties of the bio-cellulose. In some embodiments, the silver nanoparticles are blended with a gel (e.g., bio-cellulose colloid gel, bio-cellulose gel and/or hydrogel) and physically retained by the gel. In accordance with the present disclosure, the term “chemically bound” indicates that electrons are shared between the silver nanoparticles and the bio-cellulose in a covalent or ionic bond. In accordance with the present disclosure, the term “physically bound” refers to weak intermolecular forces such as, for example, coulomb forces and Van der Waals forces.

In embodiments, the final concentration of the silver nanoparticles in the bio-cellulose sheet wound dressing can be, for example, about 1600 μg/100 cm² or less, about 1500 μg/100 cm² or less, about 1250 μg/100 cm² or less, about 1000 μg/100 cm² or less, about 800 μg/100 cm² or less, about 750 μg/100 cm² or less, about 500 μg/100 cm² or less, about 400 μg/100 cm² or less, about 250 μg/100 cm² or less, about 100 μg/100 cm² or less, or about 50 μg/100 cm² or less. In embodiments, the final concentration of the silver nanoparticles in the bio-cellulose sheet wound dressing can be, for example, about 50 μg/100 cm² to about 1000 μg/100 cm². In embodiments, the MIC of the silver nanoparticles of the present disclosure allows for minimal silver nanoparticle content in the wound dressing of the present disclosure. In embodiments, the silver nanoparticles can impart a blue color forming a blue wound dressing. In embodiments, the bio-cellulose sheet including the silver nanoparticles can have a thickness of about 0.1 cm to about 1 cm.

In some embodiments, the wound dressing of the present disclosure can include a gel (e.g., bio-cellulose colloid gel, bio-cellulose gel, hydrogel or a combination of one or more thereof) in combination with silver nanoparticles. In some embodiments, bio-cellulose fiber bundles can be blended with a gelling agent to form a gel wound dressing. In embodiments, a hydrogel can be prepared by combining carboxymethyl cellulose with silver nanoparticles. In embodiments, a dilute solution of silver nanoparticles can be added to the gel during blending. In embodiments, the dilute solution of silver nanoparticles can have a concentration of about 100 mg/L of silver nanoparticles. In embodiments, a non-diluted solution of silver nanoparticles can be added the gel during blending. In embodiments, the non-diluted solution of silver nanoparticles can have a concentration of about 1000 mg/L of silver nanoparticles.

In some embodiments, the gel with silver nanoparticles can be applied directly to the wound. In accordance with some embodiments, a very thin layer of gel containing silver nanoparticles can be coated on a non-woven scaffold (e.g., sheet or gauze) or woven scaffold (e.g., sheet or gauze). The non-woven scaffold or woven scaffold can be selected from, for example, polymeric scaffolds, natural fiber scaffolds, synthetic fiber scaffolds, and the like, and combinations of these. Polymeric scaffolds can include, for example, polypropylene, polyethylene, para-aramid, polytetrafluoroethylene, poly-lactic acid, polyglycolic acid, and the like, and combinations of these. Natural fiber scaffolds can include, for example, cotton, bio-cellulose, plant cellulose, silk, viscose, and the like, and combinations of these. Synthetic fibers can include, for example, nylon, polyester, acrylic, and the like, and combinations of these. Any combination of these fibers can be used to form a non-woven scaffold or woven scaffold for the gel (e.g., bio-cellulose colloid gel, bio-cellulose gel and/or hydrogel) with silver nanoparticles. In some embodiments, the gel with silver nanoparticles can be applied to a bio-cellulose sheet that does not contain silver nanoparticles and subsequently applied to a wound.

In embodiments, the gel with silver nanoparticles can be applied directly to the wound in a thickness of about 0.1 mm to about 1.5 mm. In embodiments, the gel with silver nanoparticles in combination with the non-woven or woven scaffold can have a thickness of about 0.1 mm to about 1.5 mm.

In embodiments, the final concentration of the silver nanoparticles in the gel can be, for example, about 1600 μg/cm³ or less, about 1500 μg/cm³ or less, about 1250 μg/cm³ or less, about 1000 μg/cm³ or less, about 800 μg/cm³ or less, about 750 μg/cm³ or less, about 500 μg/cm³ or less, about 400 μg/cm³ or less, about 250 μg/cm³ or less, about 100 μg/cm³ or less, or about 50 μg/cm³ or less. In embodiments, the final concentration of the silver nanoparticles in the gel can be, for example, about 50 μg/cm³ to about 1000 μg/cm³. In embodiments, the MIC of the silver nanoparticles of the present disclosure allows for minimal silver nanoparticle content in the wound dressing of the present disclosure. In embodiments, the silver nanoparticles can impart a blue color forming a blue wound dressing.

In accordance with the present disclosure, the prepared wound dressing in sheet form and/or in a gel form can be characterized by a blue color imparted by the silver nanoparticles. In embodiments, the wound dressing can be stored in a sealed environment prior to use. In embodiments, after sealing, the wound dressing, in gel or sheet form, can be irradiated for further sterilization.

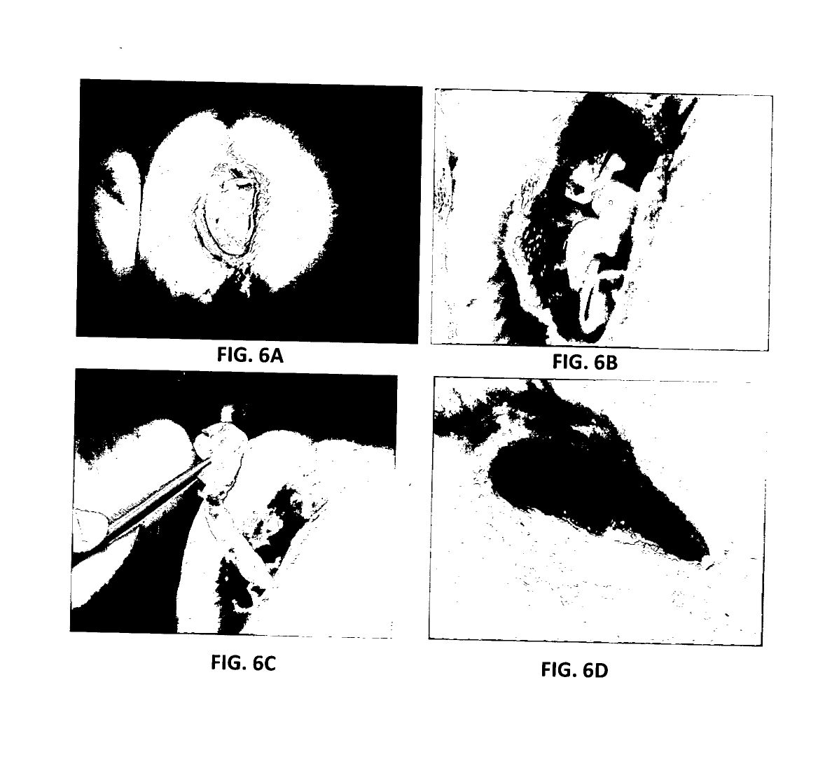

A method of application and use of a wound dressing in accordance with the present disclosure is depicted in FIGS. 6A to 6D. Depending on the type of wound to be treated, the wound dressing can be layered over the wound or loosely packed into the wound as shown in FIG. 6A, at which time the wound dressing is blue. Over time, as the silver nanoparticles impart antimicrobial benefit to the wound, the color fades to a whitish color as shown in FIG. 66. When the wound dressing has lost its blue color, a person caring for the wound can be alerted that it is time to change the dressing. As such, the wound dressing of the present disclosure has a self-indicating function or natural indicator function with respect to indicating when the wound dressing must be changed. As shown in FIG. 6C, in embodiments, the wound dressing of the disclosure can be pulled or removed from the wound in a clean manner. No obvious large tissue pieces or scabs adhere to the wound dressing of the present disclosure and the wound is not further irritated or inflamed by removal of the wound dressing of the present disclosure. As shown in FIG. 6D, following removal of the wound dressing of the present disclosure, the surface of the wound is clean and free of inflammation and scabbing.

In accordance with some embodiments, during use, the bio-cellulose of the wound dressing can protect the wound without drying the wound out. In embodiments, fluids in the wound, such as growth factors and enzymes are preserved near the wound in the bio-cellulose sheet or gel (e.g., bio-cellulose colloid gel, bio-cellulose gel and/or hydrogel). In some embodiments, the moist environment prevents scab formation over the wound base allowing new cells to migrate across the wound base to form new tissue. In embodiments, the bio-cellulose of the wound dressing facilitates auto-debridement by creating nanocapillary forces on the wound surface absorbing dead tissue and foreign matter from the wound bed. Additionally, in some embodiments, the level of pain reported by patients using the wound dressing of the present disclosure is much less than the level of pain reported by patients when using other wound dressings. Additionally, the bio-cellulose wound dressing of the present disclosure does not adhere to the wound or leave fibers behind in the wound and/or surrounding tissue.

In accordance with some embodiments, a gel (e.g., bio-cellulose colloid gel, bio-cellulose gel or hydrogel) with silver nanoparticles of the present disclosure and a bio-cellulose sheet with silver nanoparticles of the present disclosure may both be applied to the same wound for treatment of the wound. In accordance with some embodiments, a gel (e.g., bio-cellulose colloid gel, bio-cellulose gel or hydrogel) with silver nanoparticles of the present disclosure and a bio-cellulose sheet with silver nanoparticles of the present disclosure may both be applied concurrently to the same wound for treatment of the wound.

In accordance with the present disclosure, the silver nanoparticles of the wound dressing can provide an antimicrobial effect. In some embodiments, silver nanoparticles can be released (e.g., migrate or diffuse) slowly from the wound dressing to the wound site to provide antimicrobial activity and prevent microbial contamination of the moist wound site. In embodiments, the silver nanoparticles can bind with microbial proteins and matrix metallo-proteinases killing microbes, reducing inflammation at the wound area, and helping to heal the wound.

In accordance with the present disclosure, any type of wound can be treated with the wound dressings of the present disclosure. In some embodiments, wounds that can be treated include, for example, acute and/or chronic wounds, wounds in individuals with co-morbidities, burns, recurrent wounds, tunneling wounds, complicated wounds and the like, and wounds that require additional attention. In some embodiments, wounds that can be treated include tunneling wounds and complicated wounds of diabetic patients.

The wound dressing of the present disclosure can be used alone or with other dressings, bandages, and/or medicaments for treating a wound.

The present technology is further illustrated by the following examples, which should not be construed as in any way limiting.

### EXAMPLES

### Example 1

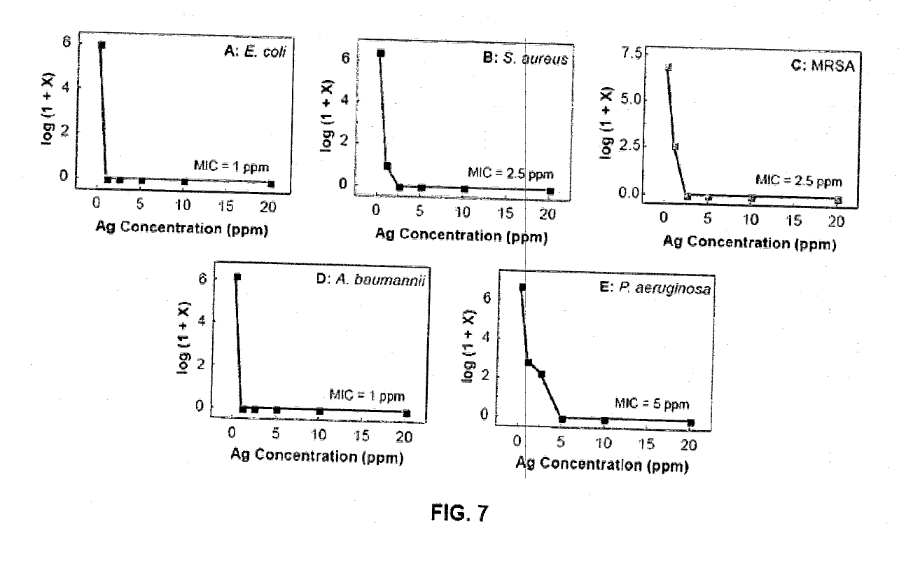

As illustrated in FIG. 7, the antimicrobial activity of blue silver nanoprisms of the present disclosure was tested against the following wound pathogens: Escherichia coli, Staphylococcus aureus, Methicillin-resistant Staphylococcus aureus (MRSA), Acinetobacter baumannii, and Pseudomonas aeruginosa. The test reports and test results are found immediately below. As demonstrated in FIG. 7 and by the results, the blue silver nanoprisms of the present disclosure exhibited strong antimicrobial activity against the wound pathogens.

A. Test Report on Antibacterial Activity of Blue Silver Nanoprisms Against Escherichia coli ATCC 25922
Test: Antibacterial activities of blue silver nanoprisms
Method: Total plate count
Bacteria Used: Escherichia coli ATCC 25922
Culture Media Nutrient agar (Difco™)

### Results

Sample: 1,000 ppm blue silver nanoprisms colloid in de-ionized water

TABLE
Antibacterial activities against Escherichia coli shown as
percent reduction of bacteria

Escherichia coli

| | |
|---|---|
| CFU/ml | % |

| Sample | 0 hr. | 24 hr. | reduction |
|---|---|---|---|
| Control (DDW) | 2.40 × 10⁶ | 1.00 × 10⁶ | 58.333 |
| Ag nano 1 ppm | 2.40 × 10⁶ | <1.0 × 10¹ | 99.999 |
| Ag nano 2.5 ppm | 2.40 × 10⁶ | <1.0 × 10¹ | 99.999 |
| Ag nano 5 ppm | 2.40 × 10⁶ | <1.0 × 10¹ | 99.999 |
| Ag nano 10 ppm | 2.40 × 10⁶ | <1.0 × 10¹ | 99.999 |
| Ag nano 20 ppm | 2.40 × 10⁶ | <1.0 × 10¹ | 99.999 |

Note:
the same data and results were obtained from a duplicate trial
CFU: colony forming units
DDW: deionized distilled water

B. Test Report on Antibacterial Activity of Blue Silver Nanoprisms Against Staphylococcus aureus ATCC 25923
Test: Antibacterial activities of blue silver nanoprisms
Method: Total plate count
Bacteria Used: Staphylococcus aureus ATCC 25923
Culture Media Nutrient agar (Difco™)

### Results

Sample: 1,000 ppm blue silver nanoprisms colloid in de-ionized water

TABLE
Antibacterial activities against Staphylococcus aureus shown as
percent reduction of bacteria

Staphylococcus aureus

| | |
|---|---|
| CFU/ml | % |

| Sample | 0 hr. | 24 hr. | reduction |
|---|---|---|---|
| Control (DDW) | 4.31 × 10⁶ | 2.65 × 10⁶ | 38.515 |
| Ag nano 1 ppm | 4.31 × 10⁶ | 1.0 × 10¹ | 99.999 |
| Ag nano 2.5 ppm | 4.31 × 10⁶ | <1.0 × 10¹ | 99.999 |
| Ag nano 5 ppm | 4.31 × 10⁶ | <1.0 × 10¹ | 99.999 |
| Ag nano 10 ppm | 4.31 × 10⁶ | <1.0 × 10¹ | 99.999 |
| Ag nano 20 ppm | 4.31 × 10⁶ | <1.0 × 10¹ | 99.999 |

Note:
the same data and results were obtained from a duplicate trial
CFU: colony forming units
DDW: deionized distilled water

C. Test Report on Antibacterial Activity of Blue Silver Nanoprisms Against Methicillin-Resistant Staphylococcus aureus (MRSA)
Test: Antibacterial activities of blue silver nanoprisms
Method: Total plate count
Bacteria Used: Methicillin-resistant Staphylococcus aureus (MRSA)
Culture Media Nutrient agar (Difco™)

### Results

Sample: 1,000 ppm blue silver nanoprisms colloid in de-ionized water

TABLE
Antibacterial activities against Methicillin-resistant
Staphylococcus aureus shown as percent reduction of bacteria

MRSA

| | |
|---|---|
| CFU/ml | % |

| Sample | 0 hr. | 24 hr. | reduction |
|---|---|---|---|
| Control (DDW) | 1.02 × 10⁷ | 8.40 × 10⁶ | 17.647 |
| Ag nano 1 ppm | 1.02 × 10⁷ | 4.15 × 10² | 99.995 |
| Ag nano 2.5 ppm | 1.02 × 10⁷ | <1.0 × 10¹ | 99.999 |
| Ag nano 5 ppm | 1.02 × 10⁷ | <1.0 × 10¹ | 99.999 |
| Ag nano 10 ppm | 1.02 × 10⁷ | <1.0 × 10¹ | 99.999 |
| Ag nano 20 ppm | 1.02 × 10⁷ | <1.0 × 10¹ | 99.999 |

Note:
the same data and results were obtained from a duplicate trial
CFU: colony forming units
DDW: deionized distilled water

D. Test Report on Antibacterial Activity of Blue Silver Nanoprisms Against Acinetobacter baumannii
Test: Antibacterial activities of blue silver nanoprisms
Method: Total plate count
Bacteria Used: Acinetobacter baumannii
Culture Media Nutrient agar (Difco™)

### Results

Sample: 1,000 ppm blue silver nanoprisms colloid in de-ionized water

TABLE
Antibacterial activities against Acinetobacter baumannii
shown as percent reduction of bacteria

Acinetobacter baumannii

| | |
|---|---|
| CFU/ml | % |

| Sample | 0 hr. | 24 hr. | reduction |
|---|---|---|---|
| Control (DDW) | 2.26 × 10⁶ | 1.54 × 10⁶ | 31.858 |
| Ag nano 1 ppm | 2.26 × 10⁶ | <1.0 × 10¹ | 99.999 |
| Ag nano 2.5 ppm | 2.26 × 10⁶ | <1.0 × 10¹ | 99.999 |
| Ag nano 5 ppm | 2.26 × 10⁶ | <1.0 × 10¹ | 99.999 |
| Ag nano 10 ppm | 2.26 × 10⁶ | <1.0 × 10¹ | 99.999 |
| Ag nano 20 ppm | 2.26 × 10⁶ | <1.0 × 10¹ | 99.999 |

Note:
the same data and results were obtained from a duplicate trial
CFU: colony forming units
DDW: deionized distilled water

E. Test Report on Antibacterial Activity of Blue Silver Nanoprisms Against Pseudomonas aeruginosa ATCC 27853
Test: Antibacterial activities of blue silver nanoprisms
Method: Total plate count
Bacteria Used: Pseudomonas aeruginosa ATCC 27853
Culture Media Nutrient agar (Difco™)

### Results

Sample: 1,000 ppm blue silver nanoprisms colloid in de-ionized water

TABLE
Antibacterial activities against Pseudomonas aeruginosa
shown as percent reduction of bacteria

Pseudomonas aeruginosa

| | |
|---|---|
| CFU/ml | % |

| Sample | 0 hr. | 24 hr. | reduction |
|---|---|---|---|
| Control (DDW) | 1.80 × 10⁷ | 5.35 × 10⁶ | 70.278 |
| Ag nano 1 ppm | 1.80 × 10⁷ | 7.10 × 10² | 99.996 |
| Ag nano 2.5 ppm | 1.80 × 10⁷ | 1.80 × 10² | 99.999 |
| Ag nano 5 ppm | 1.80 × 10⁷ | <1.0 × 10¹ | 99.999 |
| Ag nano 10 ppm | 1.80 × 10⁷ | <1.0 × 10¹ | 99.999 |
| Ag nano 20 ppm | 1.80 × 10⁷ | <1.0 × 10¹ | 99.999 |

Note:
data obtained from duplicate trial
CFU: colony forming units
DDW: deionized distilled water

### Example 2

Comparison of Calcium Alginate Wound Dressing and Wound Dressing of the Disclosure A patient having a split-thickness skin graft received treatment on ½ of the graft with a calcium alginate wound dressing and on the second ½ of the graft with a non-woven bio-cellulose sheet that did not include silver nanoparticles.

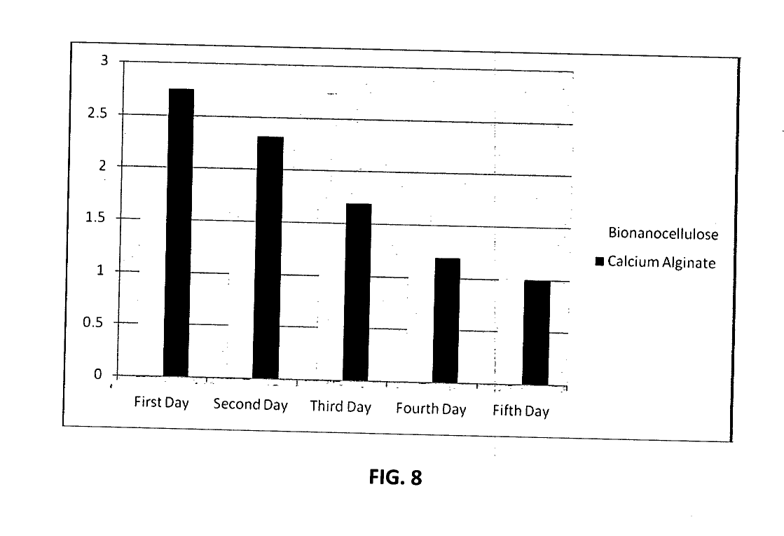

FIG. 8 depicts patient reported levels of pain on each day on each ½ of the graft. As shown in the graph, from the first day forward, the patient reported significantly lower levels of pain while using the bio-cellulose sheet.

### Example 3

### Treatment of Cavity Wound

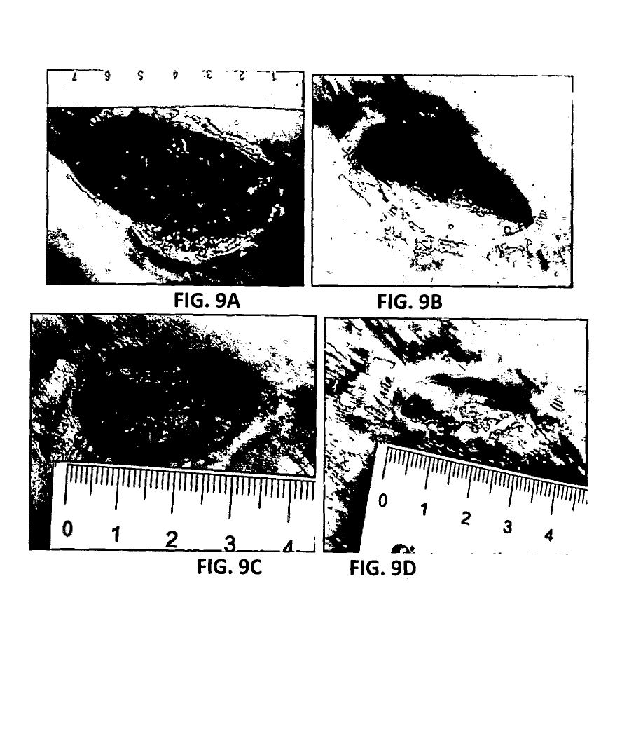

A patient presented with a cavity wound exhibiting redness, wound exudates, and inflammation. As seen in FIG. 9A, the wound was approximately 8 cm long and included two tunneling portions filled with wound exudates and inflammation.

A strip of a blue bio-cellulose sheet including silver nanoparticles in accordance with the present disclosure was placed, in a non-compacted manner, in the cavity wound. The blue bio-cellulose sheet included silver nanoparticles in a concentration of 400 μg/100 cm². During the duration of treatment, the blue bio-cellulose sheet including silver nanoparticles was replaced approximately every two to three days.

By day 14, the wound was greatly reduced in size. As seen in FIG. 9B, the amount of exudates was significantly lower. Additionally, very little inflammation was present and no infection was present. Further, there was no scabbing.

By day 28, as seen in FIG. 9C, the tunneling portions of the wound were almost closed and the wound exhibited significant healing and was reduced in size to about 4 cm.

By day 39, as seen in FIG. 9D, the wound had transformed from a cavity wound to a scar like surface wound of less than 4 cm.

### Example 4

### Dog Bite

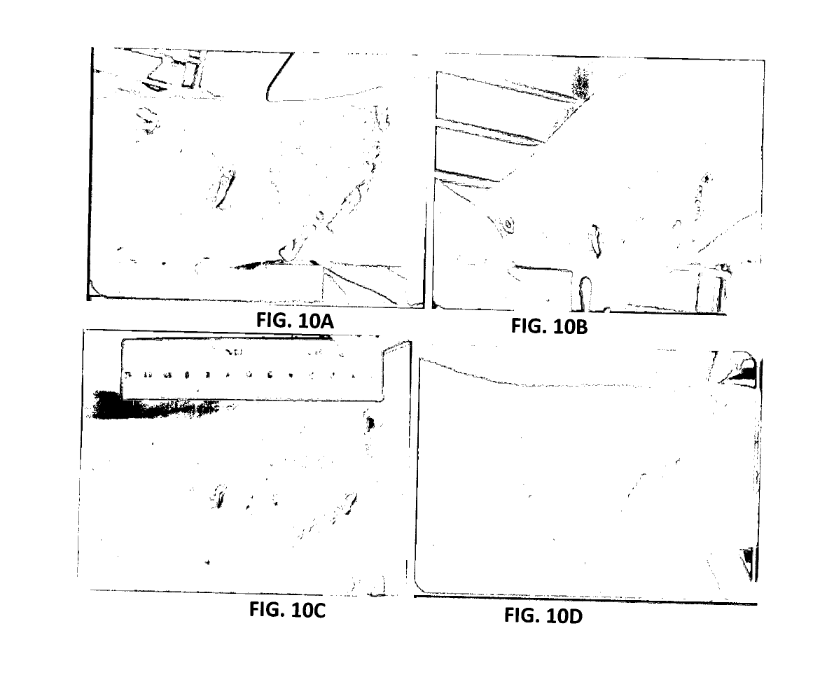

A patient presented with a severe dog bite to the arm. The lacerations included 4 major tunnels as well as multiple abrasions. As seen in FIG. 10A, each of the tunnels were gently filled (e.g., not packed in a compacted fashion) with a wound dressing of the present disclosure in the form of a blue bio-cellulose sheet including silver nanoparticles. Each blue bio-cellulose sheet used to fill each tunnel included silver nanoparticles in a concentration of 400 μg/100 cm². Additionally, a wound dressing of the present disclosure in the form of a blue bio-cellulose sheet including silver nanoparticles was used to cover the additional lacerations and abrasions. Each blue bio-cellulose sheet used to cover the additional lacerations and abrasions included silver nanoparticles in a concentration of 400 μg/100 cm². During the duration of treatment, the blue bio-cellulose sheets including silver nanoparticles were replaced approximately every two to three days.

By day 5, the tunnels of the lacerations appeared clean and free from debris and infection. As seen in FIG. 10B, no scabbing of the wound surface occurred and the tunnels were greatly reduced in depth.

On day 12, as seen in FIG. 10C, the majority of the lacerations were closed and the surface of the wounds appeared clean and not scabbed, inflamed, or infected.

By day 20, as seen in FIG. 10D, all of the lacerations had closed and the abrasions had healed. There was minimal scarring and no apparent inflammation or infection.

### Example 5

### Diabetic Callous Foot Ulcer

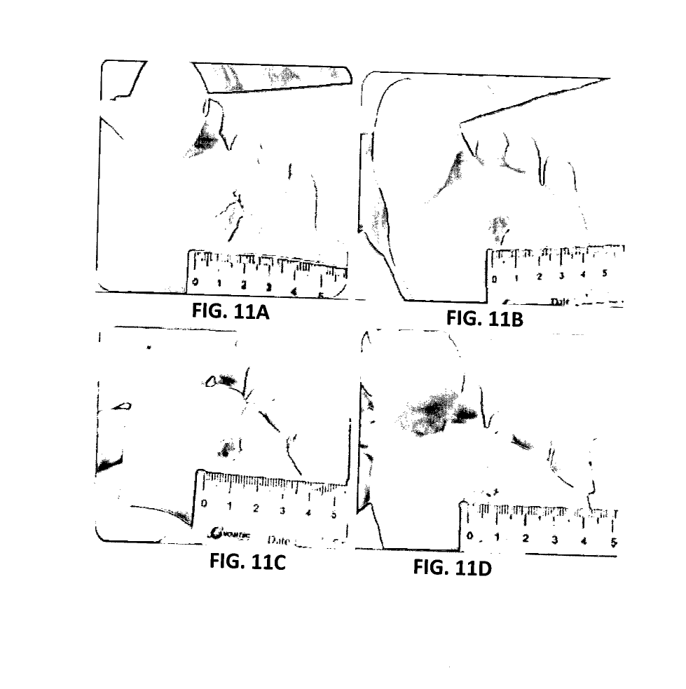

As seen in FIG. 11A, a diabetic patient presented with a thickly calloused ulcer of about 2.5 cm in length and indeterminate depth on the pad of the foot. The callous was not removed from the ulcer (as is the typical treatment method); rather a wound dressing of the present disclosure in the form of a blue bio-cellulose sheet including silver nanoparticles was used to cover the calloused ulcer. The blue bio-cellulose sheet included silver nanoparticles in a concentration of 400 μg/100 cm². During the duration of treatment, the blue bio-cellulose sheets including silver nanoparticles were replaced approximately every two to three days.

By day 14, as seen in FIG. 11B, the majority of the calloused tissue had been removed by the moisture and capillary/debriding action of the wound dressing of the present disclosure. The calloused area had reduced to about 1 cm without sharp debridement. Additionally, the ulcer present beneath the callous was evident but appeared clean.

By day 21, as seen in FIG. 11C, the size of the ulcer had reduced to about 1 cm and the ulcer appeared to be healing, clean and free of infection.

On day 42, as seen in FIG. 11D, the ulcer was about 0.5 cm in size and the calloused area had turned from yellow to a white color.

As can be seen from the above examples, the wound dressing of the present disclosure including the combination of the bio-cellulose with the silver nanoparticles in a concentration of 1000 μg/100 cm² or less provided a surprising synergistic effect in healing wounds more effectively and faster than known wound dressings.

While various aspects and embodiments have been disclosed herein, it will be apparent that various other modifications and adaptations of the disclosure will be apparent to the person skilled in the art after reading the foregoing disclosure without departing from the spirit and scope of the disclosure and it is intended that all such modifications and adaptations come within the scope of the appended claims. The various aspects and embodiments disclosed herein are for purposes of illustration and are not intended to be limiting, with the scope and spirit of the disclosure being indicated by the appended claims.

1.-20. (canceled)

21. A wound dressing comprising:
colored silver nanoparticles,
wherein the colored silver nanoparticles have a localized surface Plasmon resonance maxima of about 400 nm to about 800 nm.

22. The wound dressing of claim 21, wherein the wound dressing further comprises bio-cellulose.

23. The wound dressing of claim 21, wherein the concentration of the colored silver nanoparticles having a localized surface Plasmon resonance maxima of about 400 nm to about 800 nm is selected from the group consisting of a concentration of about 1650 μg/100 cm² or less, a concentration of about 1650 μg/cm³ or less and a combination thereof.

24. The wound dressing of claim 21, wherein the colored silver nanoparticles have a localized surface Plasmon resonance maxima of about 600 nm to about 800 nm.

25. The wound dressing of claim 21, wherein the colored silver nanoparticles have a shape selected from the group consisting of a hexagonal shape, circular disk shape, truncated triangular shape and combination of two or more thereof.

26. The wound dressing of claim 21, wherein the colored silver nanoparticles have a minimum inhibitory concentration against at least one primary wound pathogen from about 1 ppm to about 5 ppm.

27. The wound dressing of claim 26, wherein said at least one primary wound pathogen is selected from the group consisting of Escherichia coli, Staphylococcus aureus, Acinetobacter baumannii, Pseudomonas aeruginosa, and Methicillin-resistant Staphylococcus aureus.

28. The wound dressing of claim 21, wherein the bio-cellulose is a bio-cellulose sheet or the bio-cellulose is a gel selected from the group consisting of a bio-cellulose colloid gel, bio-cellulose gel and a combination thereof.

29. A method of treating a wound comprising:
providing a colored wound dressing comprising colored silver nanoparticles having a localized surface Plasmon resonance maxima of about 400 nm to about 800 nm, wherein the color of the colored wound dressing is imparted by the colored silver nanoparticles;
applying the colored wound dressing to a wound; and
removing the colored wound dressing from the wound when the colored wound dressing is a color selected from the group consisting of an off-white color, the white color of the wound dressing itself and a combination of one or more thereof.

30. The method of claim 29, wherein the wound dressing further comprises bio-cellulose.

31. The method of claim 29, wherein the method comprises:
providing a blue color wound dressing comprising bio-cellulose and colored silver nanoparticles, wherein the blue color of the blue wound dressing is imparted by the colored silver nanoparticles;
applying the blue wound dressing to a wound; and
removing the blue wound dressing from the wound when the blue wound dressing is no longer blue but instead a color selected from the group consisting of a yellowish color, off-white color, the white color of the bio-cellulose itself and a combination of one or more thereof.

32. The method of claim 29, wherein the applying step is selected from the group consisting of loosely packing the wound with the colored wound dressing, covering the wound with the colored wound dressing and a combination thereof.

33. The method of claim 29, wherein the wound is selected from the group consisting of a burn, a recurrent wound, a tunneling wound, complicated wound, a cavity wound, a dog bite wound, an ulcerous wound, a diabetic wound and a combination of one or more thereof.

34. The method of claim 29, wherein a concentration of the silver nanoparticles in the colored wound dressing is selected from the group consisting of a concentration of about 1650 μg/100 cm² or less, a concentration of about 1650 μg/cm³ or less and a combination thereof.

35. A method of preparing a colored wound dressing comprising:
adding a dilute solution of colored silver nanoparticles to a wound dressing to form a colored wound dressing, wherein the colored silver nanoparticles have a localized surface Plasmon resonance maxima of about 400 nm to about 800 nm and wherein the silver nanoparticles impart a color to the wound dressing.

36. The method of claim 35, wherein the concentration of silver nanoparticles in the colored wound dressing is selected from the group consisting of a concentration of about 1650 μg/100 cm² or less, a concentration of about 1650 μg/cm³ or less and a combination thereof.

37. The method of claim 35, wherein the wound dressing comprises bio-cellulose and the method comprises adding the dilute solution of colored silver nanoparticles to the bio-cellulose to form a colored wound dressing.

38. The method of claim 35, wherein a blue wound dressing is prepared by a method comprising:
preparing a bio-cellulose; and,
adding a dilute solution of colored silver nanoparticles to the bio-cellulose to form a blue wound dressing, wherein the concentration of the colored silver nanoparticles in the blue wound dressing is selected from the group consisting of a concentration of about 1650 μg/100 cm² or less, a concentration of about 1650 μg/cm³ or less and a combination thereof, and wherein the colored silver nanoparticles impart a blue color to the wound dressing.

39. The method of claim 35, wherein the step of preparing the bio-cellulose comprises growing Acetobacter xylinum in a media selected from the group consisting of coconut juice, pineapple juice, broken-milled rice, yeast extract and a combination of one or more thereof.

40. The method of claim 37, wherein said the step of adding the dilute solution of colored silver nanoparticles to the bio-cellulose further comprises blending the bio-cellulose and the dilute solution of colored silver nanoparticles to form a gel containing colored silver nanoparticles, wherein the gel is selected from the group consisting of bio-cellulose colloid gel, bio-cellulose gel and a combination thereof.

41. The method of claim 40, further comprising applying the gel containing colored silver nanoparticles to a scaffold selected from the group consisting of a non-woven scaffold, woven scaffold, non-woven gauze scaffold and a combination of one or more thereof.

42. The method of claim 37, wherein the step of preparing the bio-cellulose further comprises preparing a bio-cellulose sheet.

43. A wound dressing comprising a hydrogel comprising:
carboxymethyl cellulose; and
colored silver nanoparticles,
wherein the concentration of colored silver nanoparticles is about 1650 μg/cm³ or less, and wherein the silver nanoparticles have a localized surface Plasmon resonance maxima of about 400 nm to about 800 nm.

44. The wound dressing of claim 43, wherein the concentration of colored silver nanoparticles is about 1000 μg/cm³ or less, and wherein the silver nanoparticles have a localized surface Plasmon resonance maxima of about 600 nm to about 800 nm.
